(19) United States
(12) Patent Application Publication
(10) Pub. No.: US 2025/0161362 A1
(43) Pub. Date: May 22, 2025

(54) Combined preparation for its use in the improvement of the muscle health

(71) Applicant: Bioiberica, S.A.U., BARCELONA (ES)

(72) Inventors: Patricia GÁLVEZ MARTÍN, BARCELONA (ES); Javier VELASCO ÁLVAREZ, BARCELONA (ES); Daniel MARTÍNEZ PUIG, BARCELONA (ES)

(21) Appl. No.: 18/946,245

(22) Filed: Nov. 13, 2024

(30) Foreign Application Priority Data
Nov. 13, 2023 (ES) P202330926

(51) Int. Cl. A61K 35/20; A23L 33/00; A61K 31/726; A61K 31/728; A61K 38/39; A61P 21/00

(52) U.S. Cl. CPC A61K 35/20; A23L 33/40; A61K 31/726; A61K 31/728; A61K 38/39; A61P 21/00

(57) ABSTRACT

A combined preparation of two compositions acting synergistically are useful in muscular regeneration, in the treatment and/or prevention of muscular atrophy and sarcopenia in a subject, as well as for the healing of muscular wounds, improved muscular performance, of muscle senescence and in the reversal of muscular degeneration preferably associated with ageing. The compositions include hyaluronic acid, mucopolysaccharides, collagen and whey protein isolate concentrate.

The present invention relates to pharmaceutical and food compositions for use in the prevention or treatment of muscular degeneration and atrophy, preferably associated with ageing, and in particular sarcopenia. Therefore, the present invention falls within the fields of biotechnology, pharmacy and food, more particularly within the compositions intended for muscular regeneration and, in general, for the improvement of the muscular condition and performance in a subject.

### BACKGROUND OF THE INVENTION

Muscles are highly specialised structures with great potential for adaptation, largely because different signalling pathways respond to stimuli such as physical exercise. One of the effects of physical exercise is the improvement of various metabolic properties, from a reduction in circulating glucose levels, changes in muscular fibre types, to the induction of angiogenesis or regulation of the immune system. Furthermore, physical exercise prevents the development of insulin resistance and type 2 diabetes. Moreover, society is experiencing an ageing process, with an increasing proportion of people over 65 years of age. This sector of the population presents a progressive and generalised reduction of skeletal muscle, characterised by a decrease in muscular strength and physical performance, a process that is known as sarcopenia (Kalampouka, I., van Bekhoven, A. & Elliott, B. T., 2018, Front. Physiol. 9, 1-7).

Sarcopenia is associated with physical disability, falls, fractures, morbidity and mortality, poor quality of life, low bone mineral density and loss of muscular fibres (Pickering, M. E. & Chapurlat, R., 2020, Calcif. Tissue Int. 107, 203-211). The prevalence of sarcopenia in the ages of 40-49, 50-59, 60-69, 70-79 and over 80 years is 7%, 10.6%, 15.4%, 21.2% and 36.5%, respectively. The etiology of sarcopenia is multifactorial and the mechanisms underlying its pathophysiology are complex. There are several proposed mechanisms to explain the decrease in muscular mass, including cellular senescence, oxidative stress, inflammation, altered function of satellite cells, decrease in the number of motor units, changes in hormonal levels, accumulation of mutations in mitochondrial DNA or loss of myocytes by caspase-mediated apoptosis (Cheema, N., Herbst, A., Mckenzie, D. & Aiken, J. M., 2015, Aging Cell 14, 1085-1093). Traditionally, the most effective strategy for managing sarcopenia has been considered to be physical training, through resistance exercises (Cruz-Jentoft, A J et al., 2019, Age Ageing. 48, 16-31). However, many older people are sedentary and unable or unwilling to exercise, therefore new strategies are required to treat this pathology.

Numerous lines of evidence suggest a significant role of programmed cell death (apoptosis) of myocytes in sarcopenia and ageing (Quadrilatero, J., Alway, S. E. & Dupont-Versteegden, E., 2011, Appl. Physiol. Nutr. Metab. 36, 608-617). Therefore, a reduction in apoptosis may reduce the decrease in muscular mass and function in sarcopenia. Various cytokines, such as tumour necrosis factor alpha (TNF-α), interleukin 1 (IL1) or interferon-gamma (IFN-α), are involved in the pathogenesis of muscular loss. In particular, elevated levels of TNF-α have been shown to lead to muscular atrophy, since TNF-α is a potent trigger of muscular wasting in vitro and in vivo, by inhibiting myogenesis and inducing apoptosis and proteolysis, through the activation of nuclear factor-B (NF-kB) (Gallo, D. et al., 2015, Endocrinology 156, 3239-3252).

In vitro studies with myocytes are postulated as a very efficient tool to characterise the possible effects of a bioactive compound on muscular health (Yang, X., Zhang, Q., Gao, Z., Yu, C. & Zhang, L., 2018, Biomed. Pharmacother. 99, 184-190). Considering that a low myocyte renewal rate underlies the pathogenesis of aged muscle, it is reasonable to hypothesise that in in vitro muscular models, the accelerated rate of apoptosis can be induced by external signals such as TNF-α or hydrogen peroxide (H₂O₂).

In summary, novel approaches and strategies are required for the management of muscular degeneration and sarcopenia and, in general, for improving the state and performance of the muscular condition, particularly associated with ageing.

### DESCRIPTION OF THE INVENTION

The present invention provides a combined preparation comprising two compositions acting synergistically for use in muscular regeneration, in the treatment and/or prevention of muscular atrophy and sarcopenia in a subject, as well as in the healing of muscular wounds and in the reversal of muscular degeneration preferably associated with ageing.

The present invention demonstrates, by using in vitro models which simulate two different scenarios: an optimal state of health and a state of muscular degeneration, that the combined preparation to which the claims are related synergistically improves the capacity for muscular regeneration by reversing myocyte degeneration.

The exemplary embodiments shown below demonstrate the above and furthermore, to carry them out, in vitro simulation of a gastrointestinal digestion (GID) of the combined preparation of the invention has been carried out, through exposure to a series of artificial fluids, under conditions simulating what would occur in the human body after ingestion of the combined preparation of the invention, specifically to reproduce the conditions of the mouth, the stomach and intestines to which food is subjected during the digestive process, so the results shown herein can be extrapolated to what would occur in vivo.

Thus, in a first aspect, the invention relates to a combined preparation, “combined preparation of the invention”, which comprises:
a. A first composition comprising between 60 and 75% of hyaluronic acid, more than 10% of mucopolysaccharides and more than 5% of collagen (“first composition”), and
b. A second composition comprising at least 90% whey protein isolate concentrate and a fat and lactose content of less than 0.2% (“second composition”).

The term “combined preparation” refers to a preparation in which there is more than one composition, in this case the first composition indicated in (a) and the second composition indicated in (b). In the combined preparation of the invention, the two compositions do not require to be bonded to be available, but they can be separated so that their application can occur simultaneously, separately or sequentially. In this way, it is not necessarily an actual combination, in view of the fact that the two compositions can be physically separated. Preferably, the first and second compositions within the combined preparation are administered simultaneously.

In a preferred embodiment, the hyaluronic acid, the mucopolysaccharides and the collagen of the first composition are obtained from rooster comb (Gallus gallus). Thus, the first composition of the invention is, preferably, a natural extract obtained from rooster combs and comprises a concentration of hyaluronic acid between 60-75%, mucopolysaccharides (>10%) and collagen (>5%). In a more preferred embodiment, the collagen in the first composition is hydrolysed collagen. In an even more preferred embodiment, the mucopolysaccharides are chondroitin sulphate A and dermatan sulphate (chondroitin sulphate B). In another even more preferred embodiment, the first composition comprises a concentration of hyaluronic acid between 60-75%, a concentration of mucopolysaccharide of more than 10% (wherein the mucopolysaccharides are chondroitin sulphate A at a concentration between 0.40 and 3% and chondroitin sulphate B at a concentration of between 8 and 18%), a concentration of collagen of more than 5%, a concentration of fibre equal to or less than 0.4%, free amino acids at a concentration of between 0.2 and 2% and protein at a concentration of between 12 and 22%.

In a particular embodiment, and by way of examples (batches), the first composition comprises the parameters indicated below:

| Parameter | Batches | | | | | |
|---|---|---|---|---|---|---|
| (%) | 12/0001 | 12/0015 | 12/0027 | 12/0040 | FTAH04-08 | Methods |
| Hyaluronic acid | 61.7 | 63.5 | 65.6 | 69.1 | 62.1 | HPLC (Coleman et al. |
| | | | | | | 1997) and capillary |
| | | | | | | electrophoresis |
| | | | | | | (Malavaki et al., 2008) |
| | | | | | | Capillary |
| Chondroitin | 2.13 | 2.21 | 2.23 | 2.43 | 0.45 | electrophoresis |
| sulphate A | | | | | | (Malavaki et al., 2008) |
| | | | | | | Capillary |
| Chondroitin | 11.27 | 12.23 | 17.12 | 8.85 | 11.48 | electrophoresis |
| sulphate B | | | | | | (Malavaki et al., 2008) |
| Fibre | 0.4 | <0.1 | <0.1 | <0.1 | <0.2 | Weende |
| Free amino acids | 1.7 | 1.3 | 1.4 | 1.4 | 0.3 | HPLC |
| Protein | 21.4 | 18.5 | 15.5 | 18.1 | 12.6 | Lowry |
| Loss on drying | 6 | 7 | 6 | 8 | 8 | Eur. Ph. 2.2.32 |

More preferably, the process by which this first composition is obtained comprises the steps of: i) enzymatic hydrolysis of the biological samples isolated from rooster combs using a commercial alkalase produced by Bacillus licheniformis, ii) filtration, iii) concentration and iv) precipitation in the presence of NaCl. After the step of enzymatic hydrolysis, the enzymes are inactivated by means of a heat treatment.

The second composition of the invention is, preferably, a bovine whey protein isolate with a protein content between, preferably, 86 and 91%, more preferably at least 90%, with respect to dry matter, and a low lactose (<0.2%) and fat (<0.2%) content. The ash and moisture content in the second composition is less than 4.5 and 5%, respectively.

In a particular embodiment of the combined preparation of the invention, the first composition is the commercial product Mobilee® marketed by Bioibérica.

In another particular embodiment of the combined preparation of the invention, the second composition is the commercial product Lacprodan® DI-9213 marketed by Arla Food Ingredients.

The inventors of the present invention have analysed different concentrations of the first and second compositions within the combined preparation until obtaining the optimal concentrations of each of them to exert a greater effect (greater than the effect that would be obtained by adding the effects of each of the two compositions used separately) and to minimise cellular toxicity on the myocytes. Thus, in another preferred embodiment, the first composition is at a concentration of between 0.004 and 1 mg/ml of combined preparation and the second composition is at a concentration of between 0.009 and 2 mg/ml of combined preparation. In a more preferred embodiment, the first composition is at a concentration of 0.006 mg/ml of combined preparation and the second composition is at a concentration of 0.019 mg/ml of combined preparation.

The combined preparation of the invention can be in liquid, solid or semisolid (gel) form.

The combined preparation of the invention can form part of a food product or a pharmaceutical composition. To that end, another aspect of the invention relates to a food product, hereinafter, “food product of the invention”, or to a nutritional composition, comprising the combined preparation of the invention.

In a preferred embodiment of the food product or nutritional composition of the invention, said food product or nutritional composition is a food, a dietary or nutritional supplement, an ingredient of a functional food, a medicinal food (or food for special medical purposes) or a nutraceutical.

The nutritional composition or food product of the invention can be for human or animal use/consumption and refers to any food product or nutritional composition which beneficially affects the human or animal body by improving the state of muscular health in the sense indicated in the present invention.

The term “supplement” includes, although in a non-limiting manner, the terms “dietary supplement”, “nutritional supplement”, “dietary supplement”, “food supplement” and “food complement”, and refers to products or preparations the purpose of which is to supplement a subject's normal diet by providing nutrients or other substances with a physiological and/or nutritional effect. In the present invention, these substances are the components comprised in the first and second composition of the combined preparation.

Examples of food products include, but are not limited to, dietary products, plant products, meat products, snacks, beverages, cereals, bakery products, biscuits, dairy products, milks, fermented milks (such as yogurt or cheese), ice creams, butters, margarines, etc.

In another preferred embodiment, said food product is selected from the list consisting of: a beverage, including but not limited to powdered beverages, sports nutrition shakes and beverages, a dairy product, a protein bar, cereals, a cereal bar, a gel, a juice or a soup.

Another aspect of the invention relates to a pharmaceutical composition, hereinafter “pharmaceutical composition of the invention”, comprising the combined preparation of the invention.

The pharmaceutical composition of the invention can be administered to an animal, including a mammal and, therefore, to humans, in a variety of ways, including, but not limited to, oral, parenteral, intraperitoneal, intravenous, intradermal, intralesional, or intramuscular. Examples of pharmaceutical preparations include any solid composition (tablets, caplets, capsules, granules, etc.), semi-solid (gels) or liquid (solutions, suspensions or emulsions) for oral, topical or parenteral administration, preferably for oral administration. Preferably, said composition is formulated for oral administration, most preferably is administered to the subject through the diet.

In another preferred embodiment, said composition further comprises a pharmaceutically acceptable carrier and/or excipient.

The term “excipient” refers to a substance which aids the absorption of the elements of the combined preparation of the invention, stabilises said elements, activates or aids the preparation of the composition in the sense of giving it consistency or providing flavours that make it more pleasant. Thus, the excipients may have the function of holding the ingredients together such as, for example, in the case of starches, sugars, or celluloses, a sweetening function, a colouring function, a composition protection function, such as, for example, to isolate it from the air and/or humidity, the function of filling a tablet, capsule, or any other form of presentation, such as, for example, is the case of dibasic calcium phosphate, the function of disintegrating in order to facilitate the dissolution of components and the absorption thereof in the intestine, without excluding other types of excipients not mentioned herein.

The “carrier”, like the excipient, is a substance used in the composition to dilute any of the components comprised therein up to a certain volume or weight. The pharmacologically acceptable carrier is an inert substance or having an action identical to any of the elements of the present invention. The function of the carrier is to facilitate the incorporation of other elements, allow better dosing and administration or give the composition consistency and shape. When the form of presentation is liquid, the pharmacologically acceptable carrier is the diluent. The term “carrier” therefore refers to a diluent, coadjuvant, or vector with which the pharmaceutical composition of the invention must be administered; obviously, said carrier must be compatible with the compositions comprised in the combined preparation of the invention. Such pharmaceutical carriers may be liquid, such as water, solvents, oils or surfactants, including those of petroleum, animal, plant or synthetic origin, such as, for example, and not limited to, peanut oil, soy oil, mineral oil, sesame oil, castor oils, polysorbates, sorbitan esters, ether sulphates, sulphates, betaines, glycosides, maltosides, fatty alcohols, nonoxinols, poloxamers, polyoxyethylenes, polyethylene glycols, dextrose, glycerol, digitonin and the like.

As the examples below show, the combined preparation of the invention is useful for reducing the size of muscular wounds and for muscular regeneration (see FIG. 1). Furthermore, in a simulated sarcopenia situation in vitro (induced by exposure of myocytes to TNFα) this produces a strong and unexpected increase in the expression of the gene MyoD (see FIG. 2) which has a positive impact on muscular regeneration in situations of sarcopenia, showing a synergistic effect associated with the combined use of the first and second composition within the combined preparation. In summary, the combined preparation of the invention has a positive and synergistic effect on muscular regeneration and myogenesis.

Therefore, in another aspect, the invention relates to the pharmaceutical composition of the invention for use as a medicament or, alternatively, to the use of the pharmaceutical composition of the invention for preparing a medicament.

The term “medicament” refers to any substance that is used to prevent, alleviate, treat or cure diseases, syndromes or clinical conditions in humans and animals. In the context of the present invention it refers to a composition comprising the combined preparation of the invention in a therapeutically effective amount.

In the sense used in this description, the term “therapeutically effective amount” refers to the amount of the combined preparation of the invention that produces the desired effect. The dose for obtaining a therapeutically effective amount depends on a variety of factors, such as, for example, age, weight, sex or tolerance of the subject.

The medicament of the invention can be used either alone or in combination with other medicaments or compositions to promote or enhance muscular regeneration, wound healing, and the treatment and/or prevention of muscular atrophy or sarcopenia in a subject. The medicament of the present invention can be used together with other active ingredients or therapies by way of a combination therapy. The other active ingredients can be part of the same composition or can be provided by a different composition, being administered at the same time or at different times.

In another aspect, the invention relates to the pharmaceutical composition of the invention for use in muscular regeneration in a subject or, alternatively, to the use of the pharmaceutical composition of the invention for preparing a medicament to foster, enhance or induce muscular regeneration in a subject, more preferably in an ageing process and/or in a muscular wound healing process.

In another aspect, the invention relates to the pharmaceutical composition of the invention for use in the treatment and/or prevention of muscular atrophy in a subject or, alternatively, to the use of the pharmaceutical composition of the invention for preparing a medicament for the treatment and/or prevention of muscular atrophy in a subject, more preferably in an ageing process.

“Muscular atrophy” is a disorder in which muscular tissue is wasted or lost, the size of the muscle decreases, causing the same to lose strength. It is produced by an imbalance between protein synthesis and their degradation and as a consequence, nerve cells in skeletal muscles deteriorate. This causes a progressive paralysis that can be complete or partial and that implies a deterioration in the functional capacity of the person. The subject feels that his strength is diminishing and also his ability to move. A weakening, muscular shrinkage and progressive loss of muscle tone takes place. In the context of the present invention, muscular atrophy can be due to, but not limited to, physiological, pathological and neurogenic causes.

Physiological atrophy is muscular atrophy due to inactivity, due to lack of use of certain muscles as a result, for example, of leading a very sedentary life. Subjects who suffer from a disease that limits their movement or who remain seated for long periods of time, among others, are more likely to suffer from muscular atrophy.

Pathological atrophy can be caused by various diseases or occur in older people as a consequence of ageing itself.

Neurogenic atrophy, which is the most severe type of muscular atrophy and usually manifests itself more suddenly, is caused by a disease or injury to the nerves that connect the muscles. And it can appear as a consequence of diseases such as amyotrophic lateral sclerosis (ALS), due to spinal cord injury or Guillain-Barré syndrome, but it can also be a consequence of situations such as malnutrition, long-term use of corticosteroids, arthritis, muscle diseases, etc.

Another aspect of the invention relates to the pharmaceutical composition of the invention for use in the treatment and/or prevention of sarcopenia in a subject or, alternatively, to the use of the pharmaceutical composition of the invention for preparing a medicament for the treatment and/or prevention of sarcopenia in a subject.

“Sarcopenia” is a condition characterised by loss of muscular mass, strength and function in elderly subjects. Signs and symptoms include weakness, fatigue, lack of energy, balance problems and difficulties walking and standing. Loss of muscular mass or weakness sometimes causes falls, broken bones and other serious injuries that affect a person's ability to care for themselves. It is possible that advanced age, doing very little or no exercise and poor nutrition increase the risk of sarcopenia. Subjects with cancer may also present sarcopenia.

As it is used in the present invention, the term “treatment” refers to combating the effects caused as a consequence of the disease or pathological condition of interest in a subject (preferably a mammal, and more preferably human) that includes:
(i) inhibiting the disease or pathological condition, in other words, stopping its development;
(ii) alleviating the disease or pathological condition, in other words, causing the remittance of the disease or pathological condition or the symptoms thereof;
(iii) stabilising the disease or pathological condition.

As it is used in the present invention, the term “prevention” consists of preventing the onset of the disease or clinical condition, in other words, preventing the disease or pathological condition from appearing in a subject (preferably mammal, and more preferably a human), in particular, when said subject has a predisposition for the pathological condition, but has not yet been diagnosed.

The term “subject” refers to a human or a non-human animal, preferably to a non-human mammal, of any sex or age. The subject to which the present invention refers may be, for example, but not limited to, a ruminant such as a sheep, goat or cattle, dromedary, camel, llama, kangaroo, pigs, poultry such as turkey, duck, quail, goose, dove, chicken, hen or rooster, horses, as well as pets such as dogs or cats. In a particular embodiment, the subject to which the present invention refers is a human, more preferably a human over 40 years of age, preferably over 49, more than 50, more than 59, more than 60, more than 69, more than 70, more than 79, or over 80 years of age.

### DESCRIPTION OF THE FIGURES

FIG. 1. Wound size at 24 h depending on the different treatments. Data expressed as mean±SEM. * p<0.05 vs. Carrier (cell culture medium in the absence of treatments). Representative images of the regeneration capacity at the time of wounding (upper panels, 0 h) and after 24 h of treatments (lower panels). WP: Lacprodan® DI-9213.

FIG. 2. The combination of Mobilee®+Lacprodan® DI-9213 (WP) significantly and synergistically increases the expression of the MyoD gene as a marker of myogenesis in sarcopenic situation (+TNFα). Data expressed as mean±SEM.

### EXAMPLES

Next, the invention will be illustrated by means of assays carried out by the inventors which demonstrate the effectiveness of the combined preparation of the invention.

### Example 1. Experimental Design

The experimental design includes the use of in vitro cultures of L6 myocytes, a well-established skeletal muscular cell line for the study of muscular function.

Previously, the ingredients were subjected to in vitro gastrointestinal digestion, to obtain the digested fraction that was used to perform the experiments.

Next, a cell viability test was carried out to determine the range of Mobilee® concentrations tolerated by myocytes, allowing the 3 concentration levels of Mobilee® to be selected as well as the protein fraction of Lacprodan® DI-9213 (WP) (low, average and high), to finally reach those concentrations with a greater effect for the following determinations.

In order to meet the objectives set, the experiment was divided into two subsections:
S1. Myocytes were cultured in basal condition where the impact of the treatment of interest was examined in a situation simulating an optimal state of health, analysing cell migration and myogenesis.
S2. Myocytes were cultured in a situation simulating a disease condition (sarcopenia) where the impact of Mobilee® combined with WP was examined on cell migration, myogenesis and muscular atrophy.

### Example 2. Simulated Gastrointestinal Digestion In Vitro

Simulated gastrointestinal digestion in vitro (GID) is a standardised method that allows analysing changes in the physicochemical properties of an ingredient or food matrix when it is sequentially exposed to a series of artificial fluids simulating the conditions of the mouth, the stomach and small intestine. Each product was evaluated in triplicate. The GID method consists of three steps to mimic the digestive process in the mouth, the stomach (gastric digestion) and the small intestine (dynamic duodenal digestion). The human physiological environment is simulated, taking into account the conditions regarding temperature, pH, ionic strength, enzymatic activity and stirring, following the protocols described by Ortega et al. (Ortega, N., Macia, A., Romero, M. P., Reguant, J. & Motilva, M. J., 2011, Food Chem.doi:10.1016/j.foodchem.2010.05.105).

Briefly, each ingredient is mixed with a phosphate buffer saline solution (pH 6.9 with 0.04% NaCl and 0.004% CaCl₂) and then heated to 37° C. for 10 minutes. Keeping the samples under magnetic stirring and at controlled temperature (37° C.) throughout the method.
1. The oral phase was simulated by adding 10 mg of α-amylase (100-300 ud/g) in a phosphate buffer solution (pH 6.9 with 0.04% NaCl and 0.004% CaCl₂) to the different products, incubating for 5 min.
2. To reproduce the gastric phase, the pH was set at 2.0±0.1, for this purpose, a solution of porcine pepsin in 0.01 N HCl was added. This mixture was stirred in an incubator for 2 h at 37° C.
3. For duodenal digestion, the pH was adjusted to 6.5 by adding NaHCO₃. Then, duodenal juice was added, including bile salts and pancreatin and it was stirred for another 2 h at 37° C.
4. Lastly, for the dialysation process, a dialysis of 12 KDa particle size was carried out for 8 h at 37° C.

After this period, both the dialysed product and the non-dialysed portion (similar to the portion that goes to the colon) were lyophilised. Once the different digestions and dialysis were completely lyophilised, it was weighed to resuspend the digested and dialysed product for the following treatments, and be expressed as milligrams of digested and lyophilised product per millilitre of culture medium (mg/ml).

### Example 3. Cytotoxicity Assay

The cell viability test allows evaluating the range of Mobilee® concentrations tolerated by myocytes, allowing to select the different concentration levels to be tested.

Briefly, L6 cells were cultured in high glucose DMEM (Dulbecco's Modified Eagle Medium) supplemented with 10% foetal bovine serum (FBS), penicillin 100 U/ml and streptomycin solution 100 μg/ml, in a 95% humidified air atmosphere and CO₂ at 5% at 37° C. until the cells reached approximately 70-80% confluence when the treatments and different determinations were performed. Cell viability was determined using the MTT assay. Cells were treated with eight serial dilutions (¼ dilution) of the initial stock concentrations of the digested products for 24 h. After treatments, the culture medium was aspirated and the MTT solution (0.5 mg/ml) was added for 2 h. After incubation, the supernatant was replaced with DMSO (100 μl per well) and incubated for 15 min to dissolve the formazan crystals, and the absorbance was determined at 570 nm.

### Example 4. Treatment Effect in a Healthy Situation (S1) and in an In Vitro Model of Sarcopenia (S2)

The following test was carried out following the same protocol in each of the two blocks.

### Migration Test

To evaluate the effect of treatment on muscular recovery and regeneration, cell migration was analysed in basal conditions. Cells were grown to approximately an 80% confluence, the culture well was scraped with a sterile pipette tip to remove a linear section of cells. The size of the scratch was immediately photographed (initial reference), and the cells were treated for 24 h, taking microphotographs at 4 h, 24 h and 48 h after the treatments. Scratch size was quantified using ImageJ software.

Moreover, to reproduce the cellular microenvironment that occurs in sarcopenia in vitro, cells were treated with 15 ng/ml TNFα, for 24 hours and with their respective treatments.

### Determination of the Process of Myogenesis and Muscular Atrophy

Alterations in some molecular pathways may be critical in the development of both muscular damage injuries and sarcopenia. The expression of genes encoding proteins involved in the process of myogenesis or muscular atrophy was evaluated by RNA extraction and qRT-PCR. After the treatments, the medium was aspirated, cells were washed with PBS and lysed with 200 μl of TRIZOL reagent and RNA was extracted, quantifying concentration and purity using NanoDropND-1000. Contaminating DNA was removed using a DNase treatment. After RNA isolation, mRNA was reverse transcribed into cDNA using M-MLV reverse transcriptase (Invitrogen). Gene expression was analysed using a real-time quantitative PCR, using the primer set indicated below and normalised to the cyclophilin A gene PPIA, as a housekeeping gene. The differences between Cts of the studied gene and the reference gene were calculated with the 2-AACt method, obtaining relative changes in gene expression by SYBR Green fluorescence.

In particular, the analysis is carried out of the gene MyoD1 which regulates the differentiation of muscular cells and also participates in muscular regeneration.

The sequences of the oligonucleotide pair used for determining gene expression of MyoD1 are, in the 5′-3′ direction: forward primer GGAGACATCCTCAAGCGATGC (SEQ ID NO: 1) and reverse primer GCACCTGGTAAATCGGATTG (SEQ ID NO: 2).

### Example 5. Statistical Analysis

For each parameter, an initial exploration was carried out to rule out diverging points within the groups. For this purpose, the Grubbs statistical test was used, using GraphPad Prism 9 software (GraphPad Software, Inc., La Jolla, USA). All data are expressed as means±standard error of the mean (SEM). To analyse differences in the different parameters, a one-way analysis of variability (One Way ANOVA) was used. A probability level of p<0.05 was defined as statistically significant. Levels below 0.1 were defined as trends toward significance.

### Example 6. Results Simulated Gastrointestinal Digestion In Vitro

Simulated digestion in vitro of the different products is carried out at the doses indicated in Table 1.

TABLE 1
Maximum human doses and selected and escalated amounts for
simulated digestion in vitro for the different products of interest.

| Product | Amount digested |
|---|---|
| Mobilee ® | 0.8 g |
| WP | 1.5 g |

Following digestion in the different phases (oral, gastric and intestinal) the simulated intestinal absorption phase was carried out using a 12 KDa pore dialysis, obtaining, on one hand, a non-lyophilised portion resembling the colonic fraction that was stored, and on the other hand, the lyophilised portion of the digestion that resembles the fraction absorbed in the intestine, obtaining the following yields after lyophilisation (Table 2).

TABLE 2
Performance of simulated digestions in vitro of the products after
the different phases of oral, gastric and intestinal digestion, from
the simulation of the absorption process using dialysis and
from the lyophilisation of the dialysed product.

| Product | Bioaccessible fraction (%) |
|---|---|
| Mobilee ® | 23.92 |
| WP | 15.74 |

Based on the digested and lyophilisate, the cytotoxicity tests (MTT) are carried out. These tests provided a range of concentrations at which toxicity was observed and also a range of working concentrations with the different products. The cytotoxic concentrations of the digested products are shown in Table 3, and expressed as milligrams of digested, dialysed and lyophilised product per millilitre of culture medium (mg/ml).

TABLE 3
Analysis of cytotoxicity of digested products expressed as a
percentage of myocytes treated with the carrier. The concentrations
used are expressed (mg/ml) at the top of each product.

| | | 3.2 | 0.8 | 0.3 | 0.2 | 0.1 | 0.025 | 0.006 | mg/ml |
|---|---|---|---|---|---|---|---|---|---|
| Mobilee | Digested | 13.24 | 88.28 | 93.85 | 86.92 | 95.35 | 94.21 | 88.28 | % viability |

| | | 20.0 | 10.0 | 5.0 | 1.3 | 0.3 | 0.078 | 0.019 | mg/ml |
|---|---|---|---|---|---|---|---|---|---|
| WP | Digested | 2.10 | 1.69 | 6.62 | 100.20 | 100.86 | 95.19 | 93.24 | % viability |

From the cytotoxicity assays, three levels of use concentration could be extracted (high, medium and low concentration) for the digested products (Table 4) and which were applied in the following proliferation studies.

TABLE 4
Selection of high-medium-low concentrations of digested products
derived from the cytotoxicity study.

| | Concentration mg/ml | | |
|---|---|---|---|
| Product | High | Average | Low |
| Mobilee ® digested | 0.3 | 0.100 | 0.00625 |
| WP digested | 1.3 | 0.310 | 0.019 |

Next, proliferation tests were performed to verify, on the one hand, the absence of cytotoxicity with the high, average and low concentrations identified in the digested products, corroborating the absence of cytotoxicity with the three concentrations. Regarding proliferation, a trend to significantly increase proliferation was only observed in myocytes treated with the lowest concentrations (Table 5).

TABLE 5
Effects on myocyte proliferation with the different digested products
at high (upper panel), average (middle panel) and low (bottom panel)
concentrations derived from cytotoxicity studies.

| | | | Proliferation (% | |
|---|---|---|---|---|
| Concentration | Product | mg/ml | in respect of carrier) | SEM |
| High | Carrier | 0.0 | 100.00 | 4.10 |
| | Mobilee ® | 0.300 | 102.02 | 2.82 |
| | WP | 1.250 | 93.68 | 6.70 |
| Average | Carrier | 0.0 | 100.00 | 5.55 |
| | Mobilee ® | 0.100 | 101.96 | 7.34 |
| | WP | 0.310 | 98.22 | 7.83 |
| Low | Carrier | 0.0 | 100.00 | 8.10 |
| | Mobilee ® | 0.006 | 114.71 | 3.41 |
| | WP | 0.019 | 113.02 | 3.77 |

As a result of the combination of cytotoxicity and proliferation results, the usage doses of the different products and the combinations of Mobilee® with the WP were selected for the following determinations that were carried out (Table 6).

TABLE 6
Selection of the doses to be used in the following determinations
resulting from cytotoxicity and proliferation studies, expressed as
milligrams of digested, dialysed and lyophilised product per millilitre
of culture medium (mg/ml).

| Product | Dosage (mg/ml) |
|---|---|
| Mobilee ® | 0.006 |
| WP | 0.019 |
| Mobilee ® + WP | 0.006 + 0.019 |

### Cell Migration

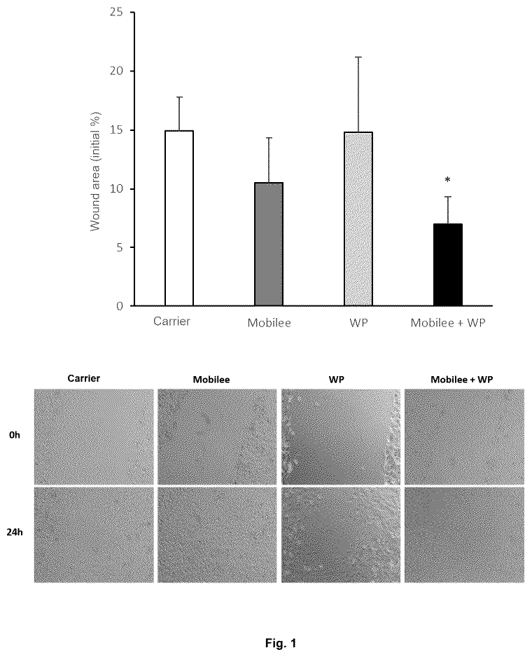

One of the most appreciated properties for muscular regeneration is the ability to reduce the size of muscular wounds. Thus, a widely accepted method is scratch, whereby a wound is made to a confluent culture of myocytes and the time it takes for complete recovery from the wound is monitored. Thus, in the present study it was observed that Mobilee® tends to reduce the size of the wound, although not significantly, WP does not produce said reduction and Mobilee® in combination with WP induces a significant reduction in the wound area (FIG. 1).

### Myogenesis

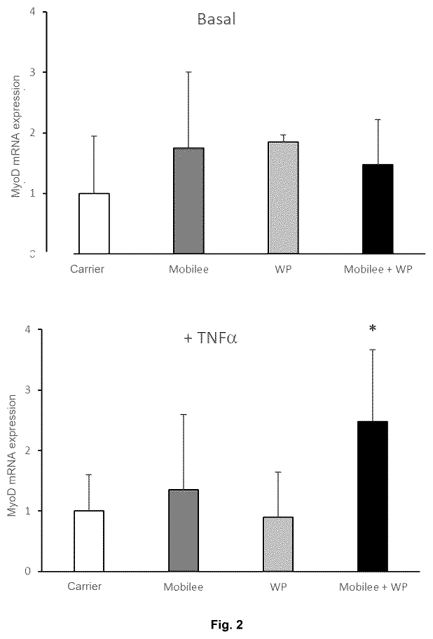

MyoD expression showed a tendency to increase with the combination of Mobilee®+WP in the baseline situation although not significantly (FIG. 2). This tendency to increase was confirmed in the sarcopenic situation produced by exposure to TNFα. Furthermore, in the sarcopenic situation due to TNFα, a strong increase in MyoD expression is observed with the combination of Mobilee® with the WP (FIG. 2), suggesting a positive and synergistic impact on muscular regeneration in situations of sarcopenia.

In conclusion, the combination proposed by the present invention (Mobilee® and WP) has beneficial effects on muscular regeneration and myogenesis in situations of sarcopenia. It can therefore be stated that treatment with the combination of Mobilee® and WP promotes muscular regeneration and myogenesis.

1. A combined preparation comprising:
a. A first composition comprising between 60 and 75% of hyaluronic acid, more than 10% of mucopolysaccharides and more than 5% of collagen, and
b. A second composition comprising at least 90% whey protein isolate concentrate and a fat and lactose content of less than 0.2%.

2. The combined preparation according to claim 1, wherein the hyaluronic acid, the mucopolysaccharides and the collagen of the first composition are obtained from rooster comb.

3. The combined preparation according to claim 1, wherein the first composition is at a concentration of between 0.004 and 1 mg/ml of combined preparation and the second composition is at a concentration of between 0.009 and 2 mg/ml of combined preparation.

4. The combined preparation according to claim 1, wherein the first composition is at a concentration of 0.006 mg/ml of combined preparation and the second composition is at a concentration of 0.019 mg/ml of combined preparation.

5. A food product comprising the combined preparation according to claim 1.

6. The food product according to claim 5, wherein said food product is a food, a dietary or nutritional supplement, an ingredient of a functional food, a medicinal food or a nutraceutical.

7. The food product according to claim 5, wherein said product is a beverage, a dairy product, a protein bar, cereals, a cereal bar, a gel, a juice or a soup.

8. A pharmaceutical composition comprising the combined preparation according to claim 1.

9. The pharmaceutical composition according to claim 8, wherein said composition is formulated for oral administration.

10. The pharmaceutical composition according to claim 8, which further comprises a pharmaceutically acceptable carrier and/or excipient.

11. (canceled)

12. (canceled)

13. (canceled)

14. (canceled)

15. (canceled)

16. A method for muscular regeneration in a subject, comprising administering to said subject the pharmaceutical composition of claim 8.

17. The method according to claim 16, wherein the subject is a human.

18. A method for treatment and/or prevention of muscular atrophy in a subject, comprising administering to said subject the pharmaceutical composition of claim 8.

19. The method according to claim 18, wherein the subject is a human.

20. A method for treatment and/or prevention of sarcopenia in a subject, comprising administering to said subject the pharmaceutical composition of claim 8.

21. The method according to claim 20, wherein the subject is a human.
